(19) United States
(12) Patent Application Publication
(10) Pub. No.: US 2022/0195481 A1
(43) Pub. Date: Jun. 23, 2022

(54) MITOCHONDRIAL RESPIROMETRY IN FROZEN SPECIMENS

(71) Applicant: The Regents of the University of Califormia, Oakland, CA (US)

(72) Inventors: Orian S. SHIRIHAI, Los Angeles, CA (US); Ilan Yaacov BENADOR-SHEN, Los Angeles, CA (US); Rebeca ACIN-PEREZ, Los Angeles, CA (US); Linsey STILES, Los Angeles, CA (US)

(73) Assignee: The Regents of the University of Califormia, Oakland, CA (US)

(21) Appl. No.: 17/610,116

(22) PCT Filed: May 15, 2020
(86) PCT No.: PCT/US2020/033112

§ 371 Date: Nov. 9, 2021

Related U.S. Application Data
(60) Provisional application No. 62/849,711, filed on May 17, 2019.

(51) Int. Cl. C12Q 1/00; G01N 33/50

(52) U.S. Cl. CPC C12Q 1/008; G01N 33/5005

(57) ABSTRACT

Mitochondrial respirometry is used to study mitochondrial functionality in healthy tissues as well as mitochondrial diseases or diseases having a link to mitochondrial function such as diabetes mellitus type 2, obesity and cancer. However, barriers to studying energy metabolism are high due to the limitations of conventional technologies which require the analysis of living or freshly isolated biological specimens. The invention disclosed herein provides a new technology to assess cellular energy production capacity in previously frozen biological specimens.

### STATEMENT AS TO RIGHTS TO INVENTIONS MADE UNDER FEDERALLY SPONSORED RESEARCH AND DEVELOPMENT

This invention was made with Government support under DK099618, awarded by the National Institutes of Health. The Government has certain rights in the invention.

### TECHNICAL FIELD

This disclosure relates to methods and materials for observing cellular energy metabolism.

### BACKGROUND OF THE INVENTION

Impaired mitochondrial respiration plays a key role in metabolic diseases, aging-related diseases, and cardiovascular disease (Liesa et al., 2009; Wallace, 2011). However, mitochondrial respirometry analysis currently requires processing and measurement of the living tissue sample within an hour of it being taken from the patient. This requirement makes respirometry analysis largely unfeasible to standard clinical practice and clinical studies. For example, a study performed in UCLA required 4 years to collect and analyze a total of 46 patient samples using current approaches (Vergnes et al., 2016). This limitation has significantly stalled scientific progress and virtually excluded the possibility to translate scientific discoveries into improved patient care.

In view of this, there is a need in the art for improved methods of observing cellular energy metabolism, specifically an ability to do so in biological samples that have been subjected to one or more freeze-thaw cycles.

### SUMMARY OF THE INVENTION

We have designed a new technology to assess cellular energy production capacity in previously frozen biological specimens, which has thus far been considered impossible by the scientific community. This technology overcomes the fundamental limitation of conventional respirometry approaches that require freshly isolated tissue and provides a simplified sample preparation which allows the use of dramatically less biological material. Moreover, this technology provides a 1,000-1,000,000-fold increase in sensitivity compared to individual complex enzymatic activity assays, the only mitochondrial assay previously possible in frozen tissue. The invention was designed following a breakthrough in our understanding of the properties of the cellular energy producing system. Briefly, the majority of cellular energy is produced by mitochondrial oxidative phosphorylation (OXPHOS), a process that harnesses the reductive energy of nutrients to power ATP biosynthesis. OXPHOS involves the sequential transfer of electrons in the inner mitochondrial membrane by specialized electron transport protein complexes. Complex I receives electrons from NADH and Complex II receives electrons from succinate. Electrons from Complex I and II subsequently flow through coenzyme Q, Complex III, cytochrome c, and Complex IV, the where oxygen is consumed in the process accepting electrons (Nicholls and Ferguson). Oxygen consumption rate, or respiration, therefore closely reflects mitochondrial electron flux.

In this context, we have discovered that the electron transport complexes assemble into respiratory active supercomplexes rather than operating as segregated enzymes (Acín-Perez et al., 2008). We have further demonstrated that supercomplexes are uniquely resistant to multiple freeze-thaw cycles unlike other components of the mitochondrial energy generating system. Building upon these discoveries, we have designed a specific combination of substrates and inhibitors that enable observations of cellular respiration in frozen biological specimens. In working embodiments of the invention, we have developed simple respirometry assays that utilize a sequence of different formulations that are combined with a sample to be assayed, formulation that contain substrates and inhibitors for Complex I, Complex II, and Complex IV. As shown in the working examples presented herein, the invention allows the measurement of electron flux in previously frozen samples for the first time.

The invention disclosed herein has a number of embodiments. One embodiment of the invention is a method for performing an assay of cellular energy metabolism in a previously frozen biological sample (e.g. one subjected to multiple freeze-thaw cycles). This method comprises the steps of combining the biological sample with a first solution comprising a substrate for Complex I, a second solution comprising a substrate for Complex II in combination with an inhibitor of Complex I, a third solution comprising an inhibitor of Complex II, a fourth solution comprising a cytochrome c reduction system, and a fifth solution comprising an inhibitor of Complex IV. Typically, these methods further comprise assaying cellular energy metabolism (e.g. by observing the oxygen consumption rate of the sample), for example in an extracellular flux analyzer device.

In embodiments of these methods, the substrate for Complex I can be nicotinamide adenine dinucleotide (NADH) and/or another substrate that donates electrons to Complex I and/or reduces Quinone. In embodiments of these methods the substrate for Complex II can be succinate and/or another substrate that donates electrons to Complex II and/or reduces quinone, and the inhibitor of Complex I comprises rotenone and/or another compound that inhibits Complex I. In embodiments of these methods, the inhibitor of Complex III can be antimycin A, myxothiazol or another compound that inhibits Complex III. In embodiments of these methods, the cytochrome c reduction system can be N,N,N′,N′-Tetramethyl-p-phenylenediamine (TMPD)/Ascorbate and/or another compound that donates electrons to Complex IV and/or reduces cytochrome c. In embodiments of these methods, the inhibitor of Complex IV can be azide, cyanide or another compound that inhibits Complex IV. Certain embodiments of the invention further comprise facilitating cellular energy metabolism by supplementing one or more of the solutions with at least one of cytochrome c, alamethicin, carbonyl cyanide-p-trifluoromethoxyphenylhydrazone, pyruvate, malate and/or N-acetylcysteine.

Another embodiment of the invention is a system for performing an assay of cellular energy metabolism in a previously frozen biological sample. This system typically comprises a first container comprising a substrate for Complex I, a second container comprising a substrate for Complex II in combination with an inhibitor of Complex I, a third container comprising an inhibitor of Complex II, a fourth container comprising a cytochrome c reduction system; and a fifth container comprising an inhibitor of Complex IV. In certain embodiments of the invention, the system includes a container comprising at least one of: cytochrome c, alamethicin, carbonyl cyanide-p-trifluoromethoxyphenylhydrazone, pyruvate, malate; and/or N-acetylcysteine. In certain embodiments of the invention, the system includes a solution having a formulation that buffers pH, chelates calcium and maintains a 270-300 mOsm/L. Optionally, the containers in this system are disposed within a kit.

Sample preparation is performed using standard procedures. In one embodiment, enzymatic digestion in combination with homogenization of the sample is employed. In one embodiment, the choice of the enzyme will depend on the tissue and animal origin. In certain embodiments, collagenase is used. In certain embodiments, trypsin is used. In certain embodiments, collagenase Type IV, trypsin collagenase Type II or nagarse is used. In one embodiment, the sample is muscle. In one embodiment, the muscle is mammalian muscle. In certain embodiments wherein the sample is muscle, collagenase Type IV, trypsin collagenase Type II or nagarse is used. In one embodiment wherein the sample is muscle, collagenase is used in combination with homogenization. In one embodiment wherein the sample is muscle, collagenase Type II is used in combination with homogenization. In one embodiment wherein the sample is muscle, trypsin collagenase Type II is used in combination with homogenization. In the zebrafish, collagenase Type IV is used. In one embodiment, collagenase is added to the homogenization buffer during the preparation of the thawed sample prior to respirometry. In some embodiments, these preparation steps avoid about a 70% reduction in respiratory rates otherwise obtained from the samples.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: A graph of data showing respiratory capacity measured in frozen isolated mitochondria using NADH instead of pyruvate and malate.

FIG. 2: A graph of data showing respiratory capacity measured in mitochondria isolated from previously frozen tissue.

FIG. 3: A graph of data showing that cytochrome C enhances respiration in frozen tissue homogenates.

FIG. 4: A graph of data showing Complex I, Complex II, and Complex IV driven respiration in frozen human adipose tissue homogenates.

FIG. 5: A graph of data showing that succinate-driven respiration is specific to Complex II in frozen mitochondria.

FIG. 6: A graph of data showing that TMPD-driven respiration is specific to Complex IV in frozen mitochondria.

FIG. 7: A graph of data showing that Complex I driven respiration in frozen isolated mitochondria treated with biguanides.

FIG. 8: A graph of data showing that respiratory capacity can be measured in frozen SH-S5Y5 neuroblastoma cells.

FIG. 9: A graph of data showing that respiratory capacity can be measured in frozen human buccal swab samples.

FIG. 10: A graph of data showing fuel preference in precursors (undifferentiated) versus differentiated macrophages.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Many of the aspects of the techniques and procedures described or referenced herein are well understood and commonly employed by those skilled in the art. The following provides a number of illustrative embodiments of the invention.

### Aspects and Elements of the Invention

Briefly, the mitochondrial respiratory chain complex of proteins is involved in oxidative phosphorylation. Oxidative phosphorylation is an important cellular process that uses oxygen and simple sugars to create adenosine triphosphate (ATP), the cell's main energy source. Five protein complexes, made up of several proteins each, are involved in this process. The complexes are named complex I, complex II, complex III, complex IV, and complex V. We have discovered that the electron transport complexes assemble into respiratory active supercomplexes rather than operating as segregated enzymes (see, e.g. Acin-Perez et al., 2008). Furthermore, we have demonstrated that supercomplexes are uniquely resistant to multiple freeze-thaw cycles unlike other components of the mitochondrial energy generating system. Building upon these discoveries, we have designed a specific combination of substrates and inhibitors that enable observations of cellular respiration in frozen biological specimens. In working embodiments of the invention, we have developed simple respirometry assays that utilize a sequence of different formulations that are combined with a sample to be assayed, formulation that contain substrates and inhibitors for Complex I, Complex II, and Complex IV.

### Illustrative Assay Protocols

Instruments Useful with Embodiments of the Invention

As discussed below, embodiments of the invention observed mitochondrial uncoupling activity by oxygen consumption rate (OCR) assay using a Seahorse XF96 Extracellular Flux Analyzer following the vendor's instructions.

While the Seahorse XF96 Extracellular Flux Analyzer is used in the working examples, analytical tools suitable for performing analysis in accordance with embodiments of the invention include, for example a Seahorse Bioscience XFp Extracellular Flux Analyzer, a Seahorse Bioscience XF^{e}24 Extracellular Flux Analyzer, a Seahorse Bioscience XF^{e}96 Extracellular Flux Analyzer, a Seahorse Bioscience XF24 Extracellular Flux Analyzer, a Seahorse Bioscience XF24-3 Extracellular Flux Analyzer, or the Seahorse Bioscience XF96 Extracellular Flux Analyzer. Each of these apparatuses enable artisans to determine a metabolic potential of a cell sample in a well of a multi-well plate. The apparatuses typically include (i) a stage adapted to support a multi-well plate; (ii) a sensor adapted to sense a cell constituent associated with the cell sample in a well of the multi-well plate; and (iii) a dispensing system adapted to introduce fluids into the well. Components of the apparatus are described in, e.g., U.S. Pat. Nos. 7,276,351 and 8,658,349, which are both incorporated, in their entireties, by reference herein.

### Sample Preparation

Thaw sample on ice. Proceed to loading the XF96 sensor cartridge.

### Loading the XF96 Sensor Cartridge

The injection ports of the XF96 Sensor Cartridge are loaded with solutions to be automatically injected during the assay. The conditions include injection of: the Complex I substrate nicotinamide adenine dinucleotide (NADH), the Complex II substrate Succinate, the Complex I inhibitor Rotenone, the Complex III inhibitor Antimycin A, the Cytochrome C reduction system N,N,N′,N′-Tetramethyl-p-phenylenediamine (TMPD)/Ascorbate, and the Complex IV inhibitor Azide (Divakaruni et al., 2014; Salabei et al., 2014). These conditions allow for the determination of the respiratory capacity of mitochondria though Complex I, Complex II, and Complex IV. The following table summarizes the order in which solutions are injected, the injection volume, and concentration, as well as the final concentration of compounds in the well to which the tissue preparations are exposed.

| XF96 | | Injection | |
|---|---|---|---|
| Port | Compounds | Volume | Final Concentration |
| A | NADH | 25 μL | 0.1-10 mM NADH |
| A | Succinate | 25 μL | 0.5-50 mM Succinate |
| | Rotenone | | 1-5 μM Rotenone |
| B | Antimycin A | 25 μL | 5-100 μM Antimycin A |
| | Rotenone | | 5-100 μM Rotenone |
| C | TMPD/Ascorbate | 25 μL | 0.5-5 mM TMPD |
| | | | 1-5 mM Ascorbate |
| D | Azide | 25 μL | 0.05-1M Azide |

Port A NADH injection is supplemented with additional reagents if maximal respiration is not reached: 1-100 μM of the chemical uncoupler carbonyl cyanide-p-trifluoromethoxyphenylhydrazone (FCCP), 0.5-50 mM pyruvate, and 0.5-50 mM malate. Port A Succinate injection is supplemented with additional reagents if maximal respiration is not reached: 1-100 μM FCCP and 0.1-50 mM of the antioxidant N-acetylcysteine. Once the ports are loaded, place the cartridge in XF96 Analyzer for calibration. Proceed to loading the XF96 Cell Culture Microplate.

### Loading the XF96 Cell Culture Microplate

All procedures are performed at 4° C. All equipment, solutions, and consumables are pre-chilled.
1. Re-suspend sample in 20 μL MAS buffer. Load 5 technical replicates of each condition. Leave at least 4 empty wells to determine background signal.
2. To adhere mitochondrial particles to the bottom of the plate, centrifuge the loaded plate at 2,000×g for 5 min at 4° C. using plate carrier rotating buckets. Do not use centrifuge break as the fast deceleration will disrupt the uniform distribution of mitochondria on the bottom of the plate.
3. After centrifugation, adjust the total volume of the wells to 150 uL by adding 130 uL MAS assay buffer per well. Supplement MAS buffer with 1-200 μg/mL cytochrome c and 1-500 μg/mL Alamethicin if maximal respiration is not reached on MAS alone. To avoid disrupting mitochondrial adherence to the bottom of the plate, add MAS using multichannel pipette pointed at a 45° angle to the top of well chamber, as instructed by the manufacturer.
4. Incubate the plate at 37° C. in a non-CO2 incubator for 5 minutes.
5. Insert plate into XF96 and begin assay protocol. Measure oxygen consumption rate (OCR) before and after every injection. Mix, wait, and measure times can vary between 1-7 minutes and 1-5 repetitions.

### Data Analysis

Export oxygen consumption rates (OCRs) from measurement instrument.

In Microsoft Excel, perform the following calculations for each individual well.
A. Complex I: Subtract minimal OCR value following Injection B Antimycin A from maximal OCR value following Injection A NADH.
B. Complex II: Subtract minimal OCR value following Injection B Antimycin A from maximal OCR value following Injection A Succinate.
C. Complex IV: Subtract minimal OCR value following Injection C TMPD from maximal OCR value following Injection D Azide.

### Solution Preparation

All solutions are prepared with the highest purity deionized water or ultra pure dimethyl sulfoxide (DMSO). pH in all aqueous solutions is adjusted to 7.2 using potassium hydroxide (KOH) and hydrochloric acid (HCl).

### Stock Solutions Stored in −20° C.

MAS buffer: 70 mM Sucrose, 220 mM Mannitol, 5 mM KH2PO4, 5 mM MgCl2, 1 mM EGTA, 0.1% BSA fatty acid-free, and 2 mM HEPES in water (or any solution that can buffer pH, chelate calcium, and maintain 270-300 mOsm/L).

Cytochrome c: 10 mg/mL in MAS.

Alamethicin: 20 mg/mL in Ethanol.

Pyruvate: 0.5 M in MAS.

Malate: 0.5 M in MAS.

Succinate: 0.5 M in MAS.

Rotenone: 40 mM in DMSO.

Antimycin: 20 mM in DMSO.

N-acetylcysteine: 1M in MAS.

Azide: 0.5M in MAS

### Solutions to Prepare on the Day of the Assay

Assay buffer: 1-200 μg/mL Alamethicin and 1-500 μg/mL cytochrome c in MAS.

NADH+Pyruvate+Malate+FCCP (10× concentration): 1-100 mM NADH (freshly prepared from powder), 5-500 mM Pyruvate, 5-500 mM Malate, 10-1,000 μM FCCP in MAS.

Succinate+Rotenone (10× concentration): 5-500 mM Succinate and 10-50 μM Rotenone in MAS.

Antimycin A+Rotenone (10× concentration): 50-1,000 μM Antimycin A and 50-1,000 μM Rotenone in MAS.

TMPD+Ascorbate (10× concentration): 5-50 mM TMPD (freshly prepared from powder) and 10-50 mM Ascorbate (freshly prepared from powder) in MAS.

Alternative Solutions and Instruments that can be Used in Embodiments of the Invention

Sample freezing: samples can be frozen before or after processing using liquid nitrogen, −80° C. freezer, −20° C. freezer, or any other freezing apparatus.

MAS buffer: Alternatives include any solution that maintains electron transport enzyme activity. This can be pure water, water supplemented with reagents that increase osmolarity, buffer pH, and/or chelate calcium. Some commonly used buffers include the following:
1. 250 M sucrose, 5-10 mM HEPES, 1-2 mM EDTA, 0.1-2.0% BSA in water.
2. 200 mM mannitol, 70 mM sucrose, 5-10 mM HEPES, 1-2 mM EDTA, 0.1-2.0% BSA in water.
3. 115 mM KCl, 10 mM KH2PO4, 2 mM MgCl, 5 mM HEPES, 1 mM EGTA, 0.1-2.0% BSA in water.
4. 250 mM sucrose, 50 mM TrisHCl (pH 8.0), 0.2 mM EDTA, 0.1-2.0% BSA in water.
5. 10 mM TrisHCl (pH 7.4), 25 mM sucrose, 75 mM sorbitol, 100 mM KCl, 10 mM K₂HPO₄, 0.05 mM EDTA, 5 mM MgCl₂, 1 mg/ml BSA.

NADH: Alternatives include any substrate that donates electrons to Complex I and/or reduces Quinone.

FCCP: Alternatives include any ionophore, such as CCCP, DNP, BAM-15 and fatty acid species.

Alamethicin: Alternatives include any membrane permeabilizing reagent, such as digitonin, saponin, or perfringolysin.

Succinate: Alternatives include any substrate that donates electrons to Complex II and/or reduces Quinone.

TMPD and Ascorbate: Alternatives include any substrate that donates electrons to Complex IV and/or reduces cytochrome c.

Rotenone: Alternatives include any compound that inhibits Complex I, such as biguanides, Stigmatellin, Piericidin, Rolliniastatin, Mucidin, Capsaicin, CoQ2 (Fato et al., 2009), Bullatacin (Miyoshi et al., 1998), ADP Ribose (Zharova and Vinogradov, 1997), acetogenins (Nakamaru-Ogiso et al., 2010), pestidices, insecticides.

Antimycin A: Alternatives include any compound that inhibits Complex III, such as Myxothiazol.

Azide: Alternatives include any compound that inhibits Complex IV, such as Cyanide.

N-acetylcysteine: Alternatives include any ROS scavenger.

Some injections can be performed simultaneously. For example, NADH and Succinate can be co-injected to assess integrated Complex I+Complex II activity. Secondly, TMPD/Ascrobate can be injected simultaneously with Rotenone/Antimycin to assess Complex IV activity. It is also possible to pre-incubate the preparation with substrates rather than inject them.

Oxygen Measurement Instruments:
1. Polarographic oxygen electrode (Clark electrode) instruments
   a. Agilent Seahorse XFe96, XFe24, XFp analyzers (Rogers et al., 2011)
   b. Oroboros Instruments O2k-FluoRespirometer
   c. Hansatech Instruments Oxygraph
2. Oxygen-sensitive fluorescent, luminescent, colorimetric, absorbance of endogenous proteins of probes
   a. Agilent Luxcel Biosciences MitoXpress Xtra
   b. NADH 340 nm absorbance, NADH 450 nm fluorescence, and cytochrome b 566 nm absorbance (Quinlan et al., 2013, 2014)
3. Spectrophotometrical mitochondrial complexes measurements (Birch-Machin and Turnbull, 2001) and in-gel activity assay (Jha et al., 2016)
4. Proton pumping sensors

### EXAMPLES

### Example 1 (20171201): Respirometry in Frozen Mitochondria Isolated from Murine Brown Adipose Tissue

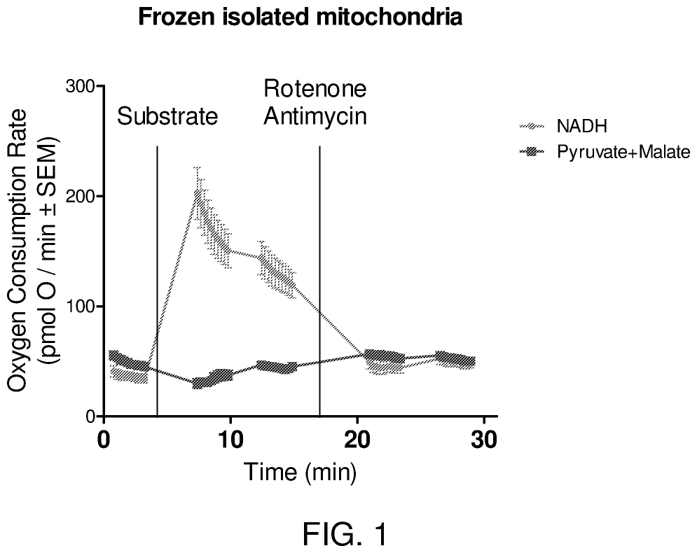

1. Mitochondria were freshly isolated from brown adipose tissue of C57/Bl6J mice as described previously in detail (Mandaviani et al., 2017).
2. 3 μg of protein was loaded per well in XF96 Cell Microplate in 20 μL MAS in 10 technical replicates.
3. The plate was centrifuged at 2,000×g for 5 min at 4° C. using plate carrier rotating buckets.
4. After centrifugation, 130 uL MAS was added per well.
5. The plate was incubated at 37° C. in a non-CO2 incubator for 5 minutes and then inserted into XF96 analyzer.
6. Seahorse assay protocol was performed with 2 minute wait, 0.3-2 minute mix, and 4 minute measure intervals and 1-2 repetitions after every injection.
7. Injection of Port A contained 10 mM NADH or 50 mM Pyruvate+50 mM Malate in MAS. Injection of Ports B contained 50 μM Antimycin and 50 μM Rotenone in MAS. Seahorse assay protocol was performed with 2 minute wait, 0.3-2 minute mix, and 4 minute measure intervals and 1-2 repetitions after every injection.
8. Results are presented in FIG. 1.

### Example 2 (20171207): Respirometry in Frozen Mitochondria Isolated from Previously Frozen Murine Heart

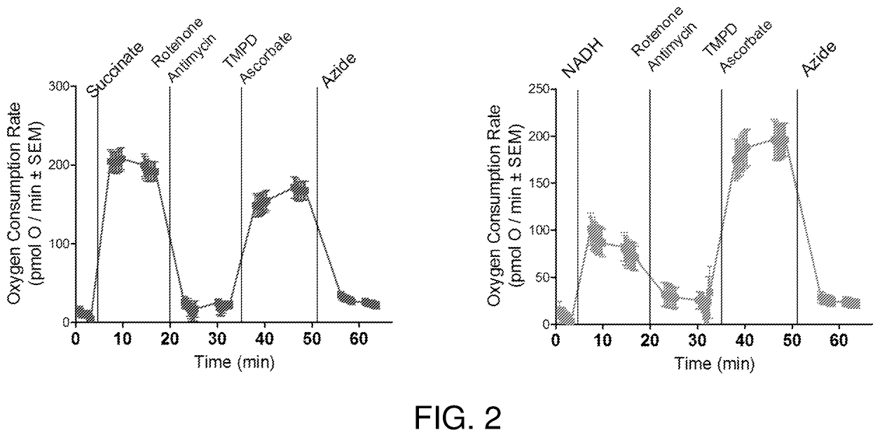

1. Mitochondria were isolated from previously frozen heart of C57/Bl6J mice as described previously in detail (Liesa et al., 2011).
2. The final mitochondrial pellet was re-suspended in ice cold isolation buffer. Mitochondrial protein was measured with a BCA assay (Pierce).
3. 2 μg of protein was loaded per well in XF96 Cell Microplate in 20 μL MAS in 5 technical replicates.
4. The plate was centrifuged at 2,000×g for 5 min at 4° C. using plate carrier rotating buckets.
5. After centrifugation, 130 uL MAS was added per well.
6. The plate was incubated at 37° C. in a non-CO2 incubator for 5 minutes and then inserted into XF96 analyzer. Injection of Port A contained 10 mM NADH or 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
7. Seahorse assay protocol was performed with 2 minute wait, 0.3-2 minute mix, and 4 minute measure intervals and 1-2 repetitions after every injection.
8. Results are presented in FIG. 2.

### Example 3 (20180112): Cytochrome C Enhances Respirometry in Frozen Murine Liver Homogenate

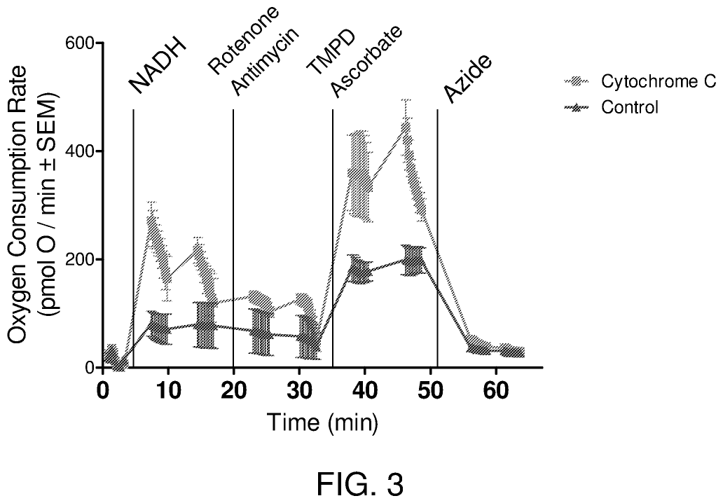

1. Homogenate was prepared from previously frozen murine liver tissue in MAS using glass/Teflon dounce homogenizer. The preparation was centrifuged at 1,000×g for 5 minutes at 4° C. and the supernatant transferred to a new tube.
2. 15 μg of protein was loaded per well in XF96 Cell Microplate in 20 μL MAS in 5 technical replicates.
3. The plate was centrifuged at 2,000×g for 5 min at 4° C. using plate carrier rotating buckets.
4. After centrifugation, 130 uL MAS or MAS supplemented with 100 μg/mL cytochrome c was added per well.
5. The plate was incubated at 37° C. in a non-CO2 incubator for 5 minutes and then inserted into XF96 analyzer. Injection of Port A contained 10 mM NADH in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
6. Seahorse assay protocol was performed with 2 minute wait, 0.3-2 minute mix, and 4 minute measure intervals and 1-2 repetitions after every injection.
7. Results are presented in FIG. 3.
This approach was validated for homogenates from frozen murine heart (4 ug), WAT (15 ug) and BAT (4 ug)

### Example 4 (20180318): Determination of Human Adipose Tissue Browning in Tissue Homogenate Prepared from Frozen Tissue of Pheochromocytoma Patients

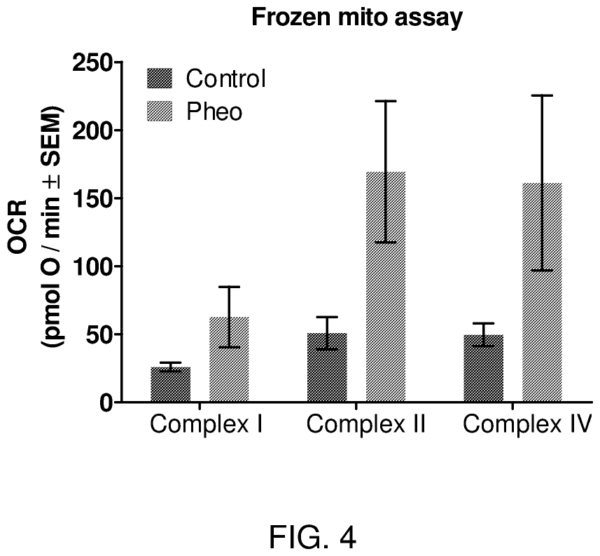

1. Adipose tissue was collected from healthy control and patients with pheochromocytoma, a hormonal disorders that increases adipose tissue bioenergetic capacity.
2. 500 mg white adipose tissue was homogenized in 2.5 mL MAS using glass/glass dounce homogenizer. The preparation was centrifuged at 1,000×g for 5 minutes at 4° C. and the supernatant transferred to a new tube.
3. 15 μg of protein was loaded per well in XF96 Cell Microplate in 20 μL MAS in 5 technical replicates.
4. The plate was centrifuged at 2,000×g for 5 min at 4° C. using plate carrier rotating buckets.
5. After centrifugation, 130 uL MAS supplemented with 100 μg/mL cytochrome c was added per well.
6. The plate was incubated at 37° C. in a non-CO2 incubator for 5 minutes and then inserted into XF96 analyzer. Injection of Port A contained 10 mM NADH or 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
7. Seahorse assay protocol was performed with 2 minute wait, 0.3-2 minute mix, and 4 minute measure intervals and 1-2 repetitions after every injection.
8. Results are presented in FIG. 4.

### Example 5 (20180223): Validation that Succinate Driven Respirations are Specific to Complex II

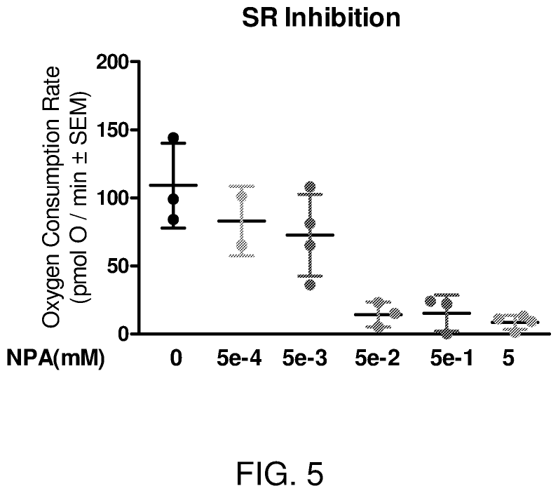

1. We used the specific Complex II inhibitor 3-Nitropropionic acid (3-NPA) to confirm the specificity of our assay.
2. 4 μg of frozen isolated liver mitochondrial protein was loaded per well in XF96 Cell Microplate in 20 μL MAS in 5 technical replicates.
3. The plate was centrifuged at 2,000×g for 5 min at 4° C. using plate carrier rotating buckets.
4. After centrifugation, 130 uL MAS or MAS supplemented with 3-NPA at the indicated concentrations was added per well.
5. The plate was incubated at 37° C. in a non-CO2 incubator for 5 minutes and then inserted into XF96 analyzer. Injection of Port A contained 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
6. Seahorse assay protocol was performed with 2 minute wait, 0.3-2 minute mix, and 4 minute measure intervals and 1-2 repetitions after every injection.
7. Results are presented in FIG. 5.

### Example 6 (20180223): Validation that TMPD Driven Respirations are Specific to Complex IV

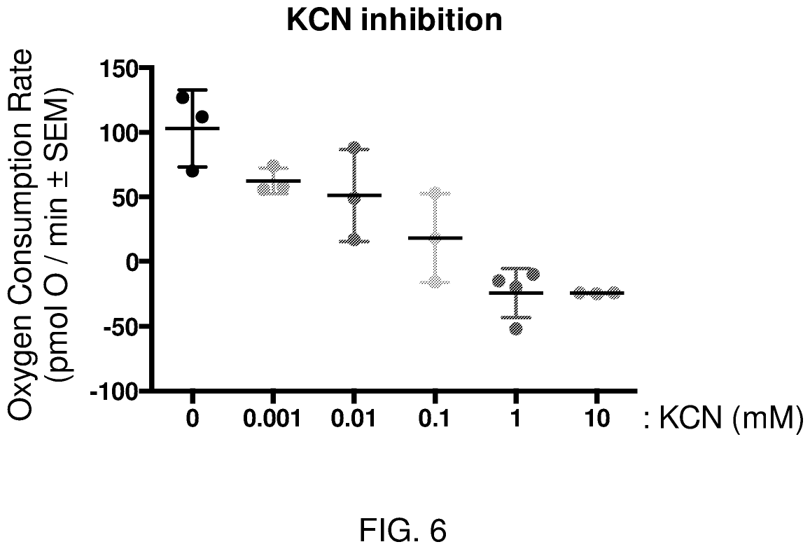

1. We used the specific Complex IV inhibitor potassium cyanide (KCN) to confirm the specificity of our assay.
2. 4 μg of frozen isolated liver mitochondrial protein was loaded per well in XF96 Cell Microplate in 20 μL MAS in 5 technical replicates.
3. The plate was centrifuged at 2,000×g for 5 min at 4° C. using plate carrier rotating buckets.
4. After centrifugation, 130 uL MAS or MAS supplemented with KCN at the indicated concentrations was added per well.
5. The plate was incubated at 37° C. in a non-CO2 incubator for 5 minutes and then inserted into XF96 analyzer. Injection of Port A contained 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
6. Seahorse assay protocol was performed with 2 minute wait, 0.3-2 minute mix, and 4 minute measure intervals and 1-2 repetitions after every injection.
7. Results are presented in FIG. 6.

### Example 7 (20180228): Determination of Mitochondrial Drug Toxicity Using Frozen Mitochondria

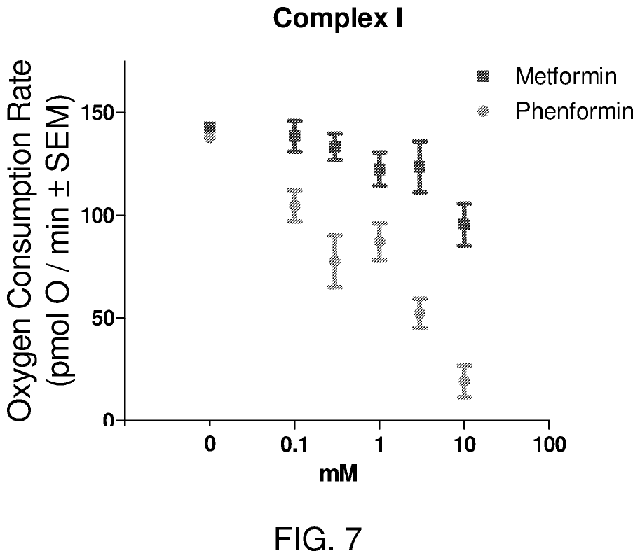

1. Metformin and Phenformin were assayed using frozen liver mitochondria isolated from murine heart. Phenformin is a biguanide that was withdrawn from the market due to Complex I toxicity resulting in lactic acidosis.
2. 4 μg of protein was loaded per well in XF96 Cell Microplate in 20 μL MAS in 5 technical replicates.
3. The plate was centrifuged at 2,000×g for 5 min at 4° C. using plate carrier rotating buckets.
4. After centrifugation, 130 uL MAS or MAS supplemented with inhibitors at the indicated concentrations was added per well.
5. The plate was incubated at 37° C. in a non-CO2 incubator for 5 minutes and then inserted into XF96 analyzer. Injection of Port A contained 10 mM NADH. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
6. Seahorse assay protocol was performed with 2 minute wait, 0.3-2 minute mix, and 4 minute measure intervals and 1-2 repetitions after every injection.
7. Results are presented in FIG. 7.

### Example 8 (20180214): Alamethicin Enhances Respiration in Frozen Cultured Human Bone Marrow Neuroblastoma Cell Line

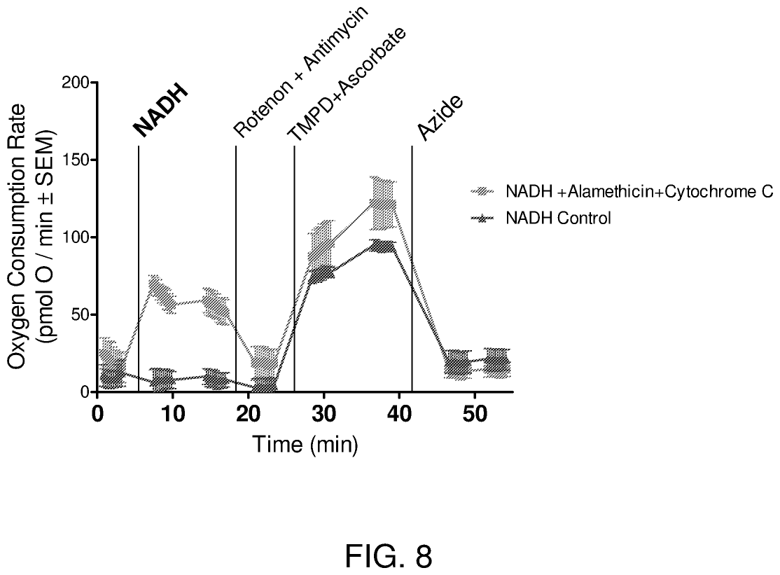

1. SH-SY5Y cells were cultured according to ATCC instructions.
2. When the cells reached 80% confluency, they were trypsinized, counted, spun down, washed in PBS, and frozen.
3. Cells were thawed on ice and re-suspended in MAS.
4. 80,000 cells were loaded per well in XF96 Cell Microplate in 20 μL MAS in 5 technical replicates.
5. The plate was centrifuged at 2,000×g for 5 min at 4° C. using plate carrier rotating buckets.
6. After centrifugation, 130 uL MAS or alone or MAS supplemented with 10 μg/mL Alamethicin and 100 μg/mL cytochrome c was added per well.
7. The plate was incubated at 37° C. in a non-CO2 incubator for 5 minutes and then inserted into XF96 analyzer. Injection of Port A contained 10 mM NADH or 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
8. Seahorse assay protocol was performed with 2 minute wait, 0.3-2 minute mix, and 4 minute measure intervals and 1-2 repetitions after every injection
9. Results are presented in FIG. 8.

### Example 9 (20180329): Respirometry in Frozen Human Buccal Mucosa Cells

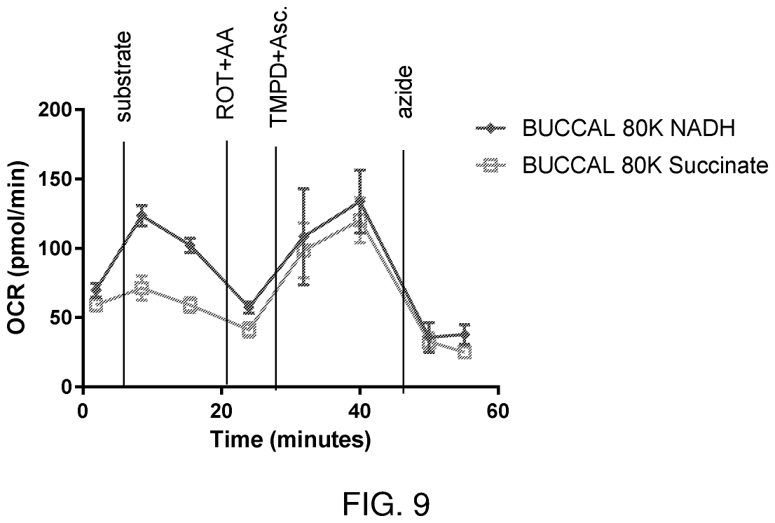

1. Human buccal mucosa cells were harvested by pressing the cotton swab in the inner cheek for 45 seconds. 4 swabs were used per subject. Each swab was clipped in individual Eppendorf tubes containing 1 ml of PBS and centrifuged at 3,000×g for 10 minutes. Cell pellets from each subject were resuspended in PBS and pooled together in one final Eppendorf tube and counted.
2. Cells were thawed on ice and re-suspended in MAS
3. 80,000 cells were loaded per well in XF96 Cell Microplate in 20 μL MAS in 5 technical replicates.
4. The plate was centrifuged at 2,000×g for 5 min at 4° C. using plate carrier rotating buckets.
5. After centrifugation, 130 uL MAS supplemented with 10 μg/mL Alamethicin and 100 μg/mL cytochrome c was added per well.
6. The plate was incubated at 37° C. in a non-CO2 incubator for 5 minutes and then inserted into XF96 analyzer. Injection of Port A contained 10 mM NADH or 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
7. Results are presented in FIG. 9.

### Example 10 (20180314): Validation of Fuel Preference Using Frozen Undifferentiated Versus Differentiated Immune Cells from Murine Bone Marrow

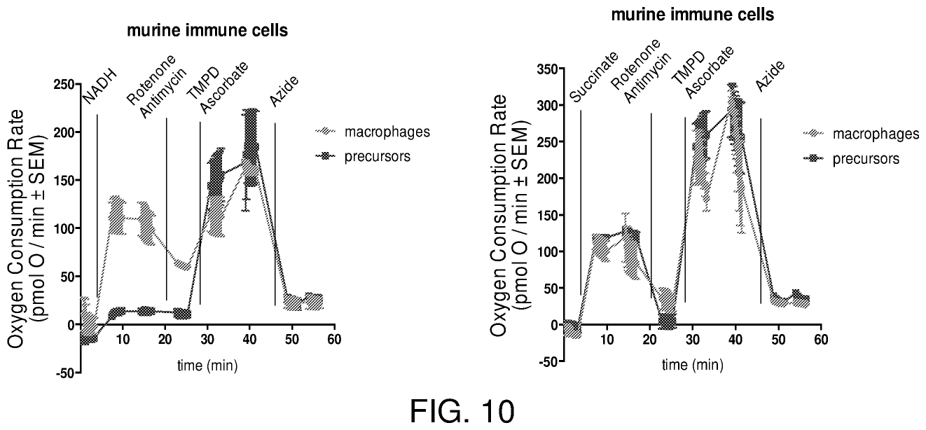

1. Murine bone marrow precursors were flushed from murine femur, cells were centrifuged at 1,000×g for 5 minutes and red blood cells lysed.
2. Precursors were plated in 10 cm dishes and grown in the presences of macrophage colony stimulation factor MCSF for 7 days to get differentiated macrophages.
3. Cell pellets from precursors and differentiated macrophages were frozen at −80° C.
4. Cells were thawed on ice and re-suspended in MAS.
5. 80,000 cells were loaded per well in XF96 Cell Microplate in 20 μL MAS in 5 technical replicates.
6. The plate was centrifuged at 2,000×g for 5 min at 4° C. using plate carrier rotating buckets.
7. After centrifugation, 130 uL MAS supplemented with 10 μg/mL Alamethicin and 100 μg/mL cytochrome c was added per well.
8. The plate was incubated at 37° C. in a non-CO2 incubator for 5 minutes and then inserted into XF96 analyzer. Injection of Port A contained 10 mM NADH or 50 mM Succinate+20 μM Rotenone in MAS. Injection of Port B contained 50 μM Antimycin and 50 μM Rotenone in MAS. Injection of Port C contained 5 mM TMPD and 10 mM Ascorbate in MAS. Injection of Port D contained 0.5M Azide in MAS.
9. Seahorse assay protocol was performed with 2 minute wait, 0.3-2 minute mix, and 4 minute measure intervals and 1-2 repetitions after every injection
10. Results are presented in FIG. 10.

### REFERENCES

Acín-Perez, R., Fernandez-Silva, P., Peleato, M. L., Pérez-Martos, A., and Enriquez, J. A. (2008). Respiratory active mitochondrial supercomplexes. Mol. Cell 32, 529-539.
Birch-Machin, M. A., and Turnbull, D. M. (2001). Chapter 5 Assaying mitochondrial respiratory complex activity in mitochondria isolated from human cells and tissues. In Methods in Cell Biology, (Academic Press), pp. 97-117.
Divakaruni, A. S., Rogers, G. W., and Murphy, A. N. (2014). Measuring Mitochondrial Function in Permeabilized Cells Using the Seahorse XF Analyzer or a Clark-Type Oxygen Electrode. Curr. Protoc. Toxicol. 60, 25.2.1-16.
Fato, R., Bergamini, C., Bortolus, M., Maniero, A. L., Leoni, S., Ohnishi, T., and Lenaz, G. (2009). Differential effects of mitochondrial Complex I inhibitors on production of reactive oxygen species. Biochim. Biophys. Acta 1787, 384-392.
Jha, P., Wang, X., and Auwerx, J. (2016). Analysis of mitochondrial respiratory chain supercomplexes using blue native polyacrylamide gel electrophoresis (BN-PAGE). Curr. Protoc. Mouse Biol. 6, 1-14.
Liesa, M., Palacin, M., and Zorzano, A. (2009). Mitochondrial dynamics in mammalian health and disease. Physiol. Rev. 89, 799-845.
Liesa, M., and Shirihai, O. S. (2013). Mitochondrial dynamics in the regulation of nutrient utilization and energy expenditure. Cell Metab. 17, 491-506.
Liesa, M., Luptak, I., Qin, F., Hyde, B. B., Sahin, E., Siwik, D. A., Zhu, Z., Pimentel, D. R., Xu, X. J., Ruderman, N. B., et al. (2011). The mitochondrial transporter ABC-me (ABCB10) is a novel gene required for cardiac recovery after ischemia-reperfusion. Circulation 124, 806-813
Mandaviani, K., Benador, I. Y., Su, S., Gharakhanian, R. A., Stiles, L., Trudeau, K. M., Cardamone, M., Enriquez-Zarralanga, V., Ritou, E., Aprahamian, T., et al. (2017). Mfn2 deletion in brown adipose tissue protects from insulin resistance and impairs thermogenesis. EMBO Rep.
Miyoshi, H., Ohshima, M., Shimada, H., Akagi, T., Iwamura, H., and McLaughlin, J. L. (1998). Essential structural factors of annonaceous acetogenins as potent inhibitors of mitochondrial complex I. Biochim. Biophys. Acta 1365, 443-452.
Nakamaru-Ogiso, E., Han, H., Matsuno-Yagi, A., Keinan, E., Sinha, S. C., Yagi, T., and Ohnishi, T. (2010). The ND2 subunit is labeled by a photoaffinity analogue of asimicin, a potent complex I inhibitor. FEBS Lett. 584, 883-888.
Nicholls, D. G., and Ferguson, S. Bioenergetics (Academic Press).
Quinlan, C. L., Perevoshchikova, I. V., Hey-Mogensen, M., Orr, A. L., and Brand, M. D. (2013). Sites of reactive oxygen species generation by mitochondria oxidizing different substrates. Redox Biol. 1, 304-312.
Quinlan, C. L., Goncalves, R. L. S., Hey-Mogensen, M., Yadava, N., Bunik, V. I., and Brand, M. D. (2014). The 2-oxoacid dehydrogenase complexes in mitochondria can produce superoxide/hydrogen peroxide at much higher rates than complex I. J. Biol. Chem. 289, 8312-8325.
Rogers, G. W., Brand, M. D., Petrosyan, S., Ashok, D., Elorza, A. A., Ferrick, D. A., and Murphy, A. N. (2011). High Throughput Microplate Respiratory Measurements Using Minimal Quantities of Isolated Mitochondria. PLOS ONE 6, e21746.
Salabei, J. K., Gibb, A. A., and Hill, B. G. (2014). Comprehensive measurement of respiratory activity in permeabilized cells using extracellular flux analysis. Nat. Protoc. 9, 421-438.
Vergnes, L., Davies, G. R., Lin, J. Y., Yeh, M. W., Livhits, M. J., Harari, A., Symonds, M. E., Sacks, H. S., and Reue, K. (2016). Adipocyte Browning and Higher Mitochondrial Function in Periadrenal But Not SC Fat in Pheochromocytoma. J. Clin. Endocrinol. Metab. 101, 4440-4448.
Wallace, D. C. (2011). Bioenergetic origins of complexity and disease. Cold Spring Harb. Symp. Quant. Biol. 76, 1-16.
Zharova, T. V., and Vinogradov, A. D. (1997). A competitive inhibition of the mitochondrial NADH-ubiquinone oxidoreductase (complex I) by ADP-ribose. Biochim. Biophys. Acta 1320, 256-264.

All publications mentioned herein (e.g. those above, Wang et al., Journal of Biomolecular Screening 2015, Vol. 20(3) 422-429 and U.S. Patent Publications 20160281049 and 20170334869) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method for performing an assay of cellular energy metabolism in a previously frozen biological sample comprising the steps of combining the biological sample with:
a first solution comprising a substrate for Complex I;
a second solution comprising a substrate for Complex II in combination with an inhibitor of Complex I;
a third solution comprising an inhibitor of Complex III;
a fourth solution comprising a cytochrome c reduction system; and
a fifth solution comprising an inhibitor of Complex IV.

2. The method of claim 1, wherein:
the substrate for Complex I comprises nicotinamide adenine dinucleotide (NADH) and/or another substrate that donates electrons to Complex I and/or reduces Quinone.

3. The method of claim 1, wherein:
the substrate for Complex II comprises succinate and/or another substrate that donates electrons to Complex II and/or reduces quinone; and/or
the inhibitor of Complex I comprises rotenone and/or another compound that inhibits Complex I.

4. The method of claim 1, wherein the inhibitor of Complex III comprises antimycin A, myxothiazol or another compound that inhibits Complex III.

5. The method of claim 1, wherein the cytochrome c reduction system comprises N,N,N′,N′-Tetramethyl-p-phenylenediamine (TMPD)/Ascorbate and/or another compound that donates electrons to Complex IV and/or reduces cytochrome c.

6. The method of claim 1, wherein the inhibitor of Complex IV comprises azide, cyanide or another compound that inhibits Complex IV.

7. The method of claim 1, further comprising assaying cellular energy metabolism by observing the oxygen consumption rate of the sample.

8. The method of claim 7, further comprising facilitating cellular energy metabolism by supplementing one or more of the solutions with at least one of:
cytochrome c;
alamethicin;
carbonyl cyanide-p-trifluoromethoxyphenylhydrazone;
pyruvate;
malate; and/or
N-acetylcysteine.

9. The method of claim 1, wherein the biological sample has been subjected to multiple freeze-thaw cycles.

10. The method of claim 7, wherein the assay of cellular energy metabolism is performed in an extracellular flux analyzer device.

11. A system for performing an assay of cellular energy metabolism in a previously frozen biological sample comprising:
a first container comprising a substrate for Complex I;
a second container comprising a substrate for Complex II in combination with an inhibitor of Complex I;
a third container comprising an inhibitor of Complex III;
a fourth container comprising a cytochrome c reduction system; and
a fifth container comprising an inhibitor of Complex IV.

12. The system of claim 11, further comprising a container comprising at least one of:
cytochrome c;
alamethicin;
carbonyl cyanide-p-trifluoromethoxyphenylhydrazone;
pyruvate;
malate; and/or
N-acetylcysteine.

13. The system of claim 11, further comprising a solution having a formulation that buffers pH, chelates calcium and maintains a 270-300 mOsm/L.

14. The system of claim 11, wherein the containers are disposed within a kit.

15. The system of claim 11, further comprising a sensor cartridge.

16. The method of claim 1 wherein the sample is muscle, liver, heart, buccal mucosa cells, adipose tissue, mitochondria or bone marrow.

17. The method of claim 1 wherein prior to combining, the sample is subjected to homogenization.

18. The method of claim 17 wherein the sample is also subjected to enzymatic digestion.

19. The method of claim 18 wherein the enzymatic digestion comprises collagenase.

20. The method of claim 18 wherein the enzymatic digestion comprises collagenase Type II, collagenase Type IV, trypsin collagenase Type II or nagarse.

21. The method of claim 19 wherein the sample is muscle.

22. The method of claim 21 wherein collagenase Type II is used.

23. The method of claim 21 wherein collagenase Type IV is used.

24. The system of claim 11 further comprising a container comprising an enzyme for digesting the sample.

25. The system of claim 24 wherein the enzyme is collagenase.

26. The system of claim 24 wherein the enzyme is selected from collagenase Type II, collagenase Type IV, trypsin collagenase Type II or nagarse.
